(19) United States
(12) Plant Patent Application Publication
(10) Pub. No.: US 2021/0321551 P1
(43) Pub. Date: Oct. 14, 2021

(54) Mentha Canadensis plant named 'Umpqua'

(50) ;

(71) Applicant: WILD Flavors, Inc., Erlanger, KY (US)

(72) Inventors: Jie Liu, Corvallis, OR (US); Deven Holmgren, Eugene, OR (US); Dan Brosten, Kalispell, MT (US); Tim Nemeth, Kalamazoo, MI (US); Tim Chambers, Kalamazoo, MI (US); Mark Morris, Euguene, OR (US); A.J. Todd, IV, Kalamazoo, MI (US); Rich Schneider, Sunnyside, WA (US); Mark Nelson, Caldwell, ID (US); Bryon Quebbeman, La Grande, OR (US)

(21) Appl. No.: 16/846,147

(22) Filed: Apr. 10, 2020

(51) Int. Cl. A01H 6/50

(52) U.S. Cl. PLT/259

(57) ABSTRACT

A new and distinct variety of Mentha canadensis, ‘Umpqua’ is characterized by its unique flavor profile providing for a distinct long-lasting mouth cooling sensation. The new varietal offers a very clean, fresh mouthfeel and intense nasal properties with strong cooling characteristic sensations that increase as it is experienced.

Latin name of the genus and species of the plant claimed: Mentha canadensis.

Variety determination: ‘Umpqua’.

### FIELD OF THE INVENTION

The present invention relates to a new and distinct mint plant botanically known as Mentha canadensis. The new variety has been named ‘Umpqua’ and will be referred to as such herein.

### BACKGROUND OF THE INVENTION

The essential oil industry refers to members of this species as “Mentha arvensis”. This includes varieties as ‘Shivalik’, ‘Kosi’, ‘Kalka,’ etc. Botanically, these plants are M. canadensis species, with Mentha arvensis being a different, significantly less commercial, mint species. The botanical differences allow one to differentiate between the two species through the use of current botanical keys for the genus Mentha (Lawrence 2007; Tucker and Debaggio 2000). The use of “Mentha arvensis” by the industry should be interpreted as a colloquial industry usage for an “oil type” and not one of botanical specificity as these plants are correctly botanically identified as M. canadensis.

### SUMMARY OF THE INVENTION

The new mint plant of the present invention was developed in an effort to cultivate a mint variety having a unique flavor profile providing for a distinct long-lasting mouth cooling sensation compared to other mint plants. The new varietal offers a very clean, fresh mouthfeel and intense nasal properties with strong cooling characteristic sensations that increase as the oil from the new varietal is experiences.

Umpqua oil may be used in flavoring confections, pharmaceuticals, beverages, tobacco, and oral care products including, but not limited to, chewing gum, hard boiled candy, soft chews, pressed tablets, mint flavored chocolate, bakery, toothpaste, mouthwash, cough and throat lozenges, cigarettes, smokeless tobacco, flavored beverages, and syrups. The oil from Umpqua may be used at usage levels from 0.01 to 5.0% by weight, either by itself or in combination with other mint oils, components thereof, or natural and/or synthetic flavoring materials in the finished product depending on the finished application.

Umpqua is disease resistant, has a greater stand longevity as compared to other mint plants, demonstrates excellent growth vigor, requires less crop rotation, and provides greater yields that other mint plants, while enhancing the cooling sensory experiences in flavor profiles. These qualities give mint growers a more sustainable and profitable option over the typical M. canadensis varieties grown.

The inventors explored non-GMO methods of creating genetically distinct mint plants through soma clonal variation of the variety of Mentha canadensis Metolius described in U.S. Plant Pat. No. 21,053 P3. Umpqua was created using this method.

Umpqua has been under continuous evaluation. Asexual propagation though stem and rhizome cuttings have been successfully carried out many times. The resulting propagules have remained stable in appearance, oil quality, and other characteristics to the original planta.

The present invention relates to a new and distinct mint plant named ‘Umpqua’ characterized by a unique flavor profile providing a long-lasting, mouth cooling sensation compared to other mint plants. The oil chemistry and flavor profile of ‘Umpqua’ is also similar throughout its maturity when grown in the U.S. mint producing regions of Oregon, Washington, and Idaho.

### BRIEF DESCRIPTION OF THE DRAWINGS

The accompanying color photographs of ‘Umpqua’ shows the new variety.

FIG. 1 shows the flower spike of ‘Umpqua.’

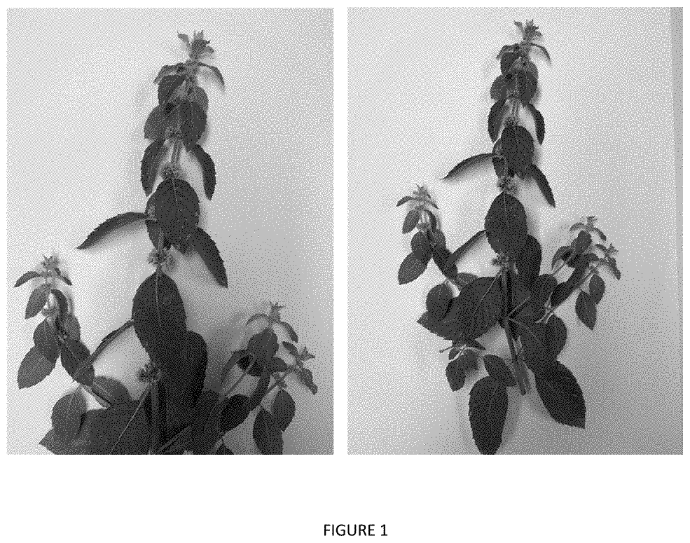

FIG. 2 shows the mature leaves of ‘Umpqua.’

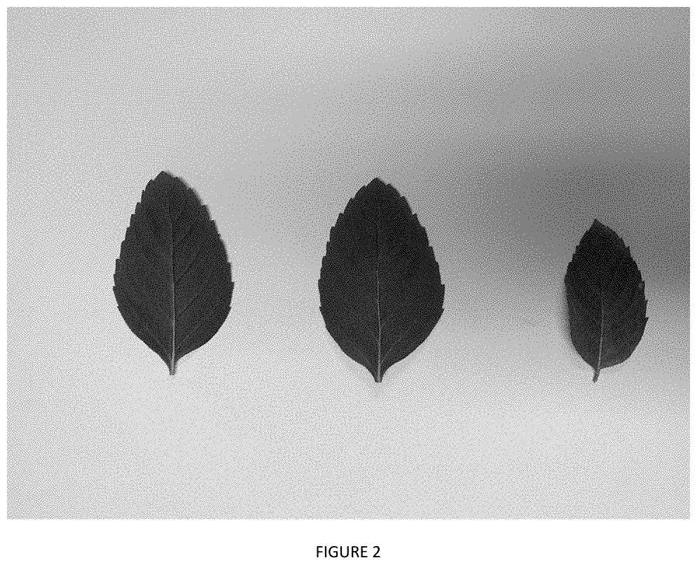

FIG. 3 shows SNPs of Umpqua compared to other Mentha canadensis varieties.

### DETAILED DESCRIPTION OF THE INVENTION

The present invention is a new and distinct variety of a Mentha canadensis plant having the following characteristics that, in combination, are not exhibited in other M. canadensis plants:
1. oil having a distinct flavor profile characterized by significantly longer lasting mouth and nasal cooling compared to other mint plants along with a clean profile;
2. oil characterization for this M. canadensis varietal is featured having typical chemistry markers represented by the following ranges for Menthone 5.0 to 25.0%, d-Isomenthone 3.0 to 6.0%, isopulegol 0.3 to 0.8%, neo-menthol 1.0 to 3.0%, Menthol 50 to 80%, Pulegone 0.0 to 0.2%, beta-Caryophyllene 0.4 to 1.0% and Germacrene D 0.5 to 2.5%; and
3. strong resistance to the diseases Verticillium wilt and mint rust.

Oil Composition. The analytical assay determined that ‘Umpqua’ has essentially the same chemical composition structure as other M. canadensis cultivars with identical components, the relative amounts feature components as discussed herein that follow within range of a typical canadensis with very little variation, but complete distinction from other known cultivars of mint varieties such as M. x piperita.

Analytical results show the presence of typical molecular markers found in M. canadensis, like alcohols, ketones, monoterpenes and sesquiterpenes. When the essential oil from this NA canadensis variety is steam distilled, the oil yields a mix of alcohols such as isopulegol (0.3 to 0.8%), neo-Menthol (1.0 to 3.0%) and Menthol (50 to 80%), ketones such as Menthone (5.0 to 25.0%), d-Isomenthone (3.0 to 6.0%), Pulegone (0.0 to 0.3%) and Piperitone (0.2 to 0.6%), monoterpenes such as l-Limonene (1.0 to 4.5%), and sesquiterpenes such as beta-Caryophyllene (0.4 to 1.0%) and Germacrene D (0.5 to 2.5%).

The oil chemistry and flavor profile of ‘Umpqua’ is sustained throughout its maturity when grown in U.S. mint producing regions.
Botanical description: M. canadensis ‘Umpqua’ is an herbaceous perennial with an upright shrubby growth habit. The plant spreads by stolons. It is reasonable for a single rooted cutting to spread and cover 3-4 square feet after one growing season. Numerous branching stems are produced each year with an eventual height of 70-80 cm.
Stem and leaves: Umpqua has a square stem, 7-8 mm on a side. Stems and lanceolate leaves are pubescent, oppositely arranged on the stem with an equilateral base, an acute apex, serrate margin, and decreasing in size towards the apex of a blooming stem. Leaf surface color is medium green, with the ventral leaf surface, petiole, and stem being a slightly lighter green color. The base of the stem has a more “woody” appearance and quality with more brownish coloring and fissures. The fragrance is clean mint-like.
Flower: Umpqua possesses a flower spike with a pair of bract-like leaves subtending each verticillaster. The individual flowers contain a 4-lobed, nearly regular mostly white corolla with a short tube, 8 mm long from the base of the calyx to the tip of the forked white stigma. The calyx generally has an average of five teeth fused at the base forming a short tube and is 3 mm long. The calyx, peduncle and pedicel colors are light yellow green. Each flower has one pistil with an 8 mm long white style and four epipetalous stamens that are exerted with 4 mm long white filaments. The white anthers are less than 1 mm. The pollen color is yellow and the formation of seed is a rare event. There is no obvious flower fragrance.

The above description is based on observations of a field plot made during the middle of its second growing season outdoors in western Oregon.

A single-nucleotide polymorphism (SNP) is a substitution of a nucleotide that occurs at a specific position in the genome. SNPs are the most common form of genome variation. SNPs are abundant and widely distributed within the genome. Genotyping by sequencing (GBS) is a method to discover SNP in order to perform genotyping studies. Mint genomic DNA was extracted and digested using restriction enzyme ApeKI. Next generation sequencing was performed resulting in about 100 bp single end reads. Raw sequence data are filtered and are aligned to Mentha longifolia genome as a reference. A total of 207,309 quality SNPs was found and selected from 15 M. x piperita, M. canadensis, and M. aquatica varieties by using GBS.

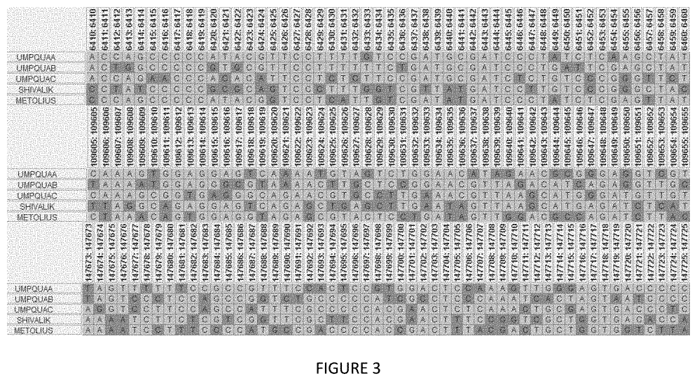

Examples of SNPs among the 5 M. canadensis varieties are shown in FIG. 3. It revealed 19 SNPs at physical positions 6410, 6425, 6434, 6435, 6440, 6441, 109606, 109611, 109623, 109635, 109636, 109651, 147675, 147676, 147682, 147708, 147709, 147716, 147725 distinguishing 3 populations of Umpqua from Shivalik and Metolius. For example, from the positions 6440-6441 which reads GC for Umpqua vs AT for Shivalik and Metolius; from the positions 109635 to 109636 which reads AC for Umpqua vs TA for Shivalik and Metolius.

Above genetic description based on SNP markers indicated a close genetic relationship between Umpqua with other M. canadensis varieties and the unique genetic profiles of Umpqua from other M. canadensis varieties and these SNP variations are within the different varieties of the same mint species M. canadensis.

Umpqua has been grown in different field locations. Umpqua has also been grown under greenhouse conditions. The basic morphological characteristics have remained consistent, with minor differences easily attributed to differences in climate, soils, fertilizer, water regime, etc. Umpqua remains distinct from other mints in its characteristics.

What is claimed is:

1. A new and distinct variety of Mentha canadensis named ‘Umpqua’ substantially as shown and described.
